(19) United States
(12) Patent Application Publication
(10) Pub. No.: US 2024/0358774 A1
(43) Pub. Date: Oct. 31, 2024

(54) USE OF MICROBIOME IN THE FIELD OF MICROBIALS AND THERAPEUTICS

(71) Applicant: CARDIAI TECHNOLOGIES, Calgary (CA)

(72) Inventors: Minal Borkar Tripathi, Calgary (CA); Sidharth Singh Bhinder, Calgary (CA); Anmol Singh Kapoor, Calgary (CA); Amrita Kaur, Calgary (CA)

(21) Appl. No.: 18/646,922

(22) Filed: Apr. 26, 2024

Related U.S. Application Data
(60) Provisional application No. 63/498,608, filed on Apr. 27, 2023.

(51) Int. Cl. A61K 35/741; A61K 36/06

(52) U.S. Cl. CPC A61K 35/741; A61K 36/06

(57) ABSTRACT

The present disclosure relates to a method for modulating microbiome activity in an individual to treat diseases and disorders by administering an effective amount of a microbiome-modulating agent selected from the group consisting of antimicrobial agents, probiotic agents, prebiotic agents, or combinations thereof. The administering is performed orally, intranasally, intramuscularly, intravenously or topically or any other suitable route, Further, the microbiome-modulating agent is administered in an amount effective to modulate microbiome activity.

### CROSS-REFERENCE TO RELATED APPLICATIONS

This application claims the benefit of U.S. Provisional Application No. 63/498,608 titled “USE OF MICROBIOME IN THE FIELD OF MICROBIALS AND THERAPEUTICS” filed by the applicant on Apr. 27, 2023, which is incorporated herein by reference in its entirety.

### FIELD OF THE INVENTION

Embodiments of the present invention relate to the field microbiome therapeutics and more particularly, relate to a method for modulating microbiome activity to treat diseases and disorders.

### DESCRIPTION OF THE RELATED ART

A microbiome is an ecological community of commensal, symbiotic, and pathogenic microorganisms that are associated with an organism. The human microbiome comprises as many microbial cells as human cells present in the entire human body, but characterization of the human microbiome is still in nascent stages due to limitations in sample processing techniques, genetic analysis techniques, and resources for processing large amounts of data.

The microbiome is a complex ecosystem of microorganisms that inhabit various parts of the human body, including the skin, mouth, gut, and reproductive tract. In particular, the human microbiome, comprising trillions of microorganisms residing in and on the body, plays a crucial role in maintaining health and regulating various physiological functions. These microorganisms include bacteria, viruses, fungi, and other microbes.

It is recognized that mammals are colonized by microbes in the gastrointestinal (GI) tract, on the skin, and in other epithelial and tissue niches such as the oral cavity, eye surface and vagina. The gastrointestinal tract, vagina and other niches harbor an abundant and diverse microbial community. It is a complex system, providing an environment or niche for a community of many different species or organisms, including diverse strains of bacteria. Hundreds of different species may form a commensal community in the GI tract or vagina of a healthy person, and this complement of organisms evolves from the time of birth to ultimately form a functionally mature microbial population by about 3 years of age. A substantial diversity of species may form a commensal community in the gut and the vagina in a healthy person. Interactions between microbial strains in these populations, and between microbes and the host, e.g. the host immune system, shape the community structure as well as microbial niches distal to the intestinal lumen, with availability of and competition for resources affecting the distribution of microbes. Such resources may be food, location and the availability of space to grow or a physical structure to which the microbe may attach. For example, the host diet is involved in shaping the GI tract flora and vaginal flora.

A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. In settings of ‘dysbiosis’ or disrupted symbiosis, microbiota functions can be lost or deranged, resulting in increased susceptibility to pathogens, altered metabolic profiles, or induction of proinflammatory signals that can result in local or systemic inflammation or autoimmunity. Thus, the intestinal microbiota plays a significant role in the pathogenesis of many diseases and disorders, including a variety of pathogenic infections distal to the gastrointestinal tract.

Recent research has unveiled that the microbiome plays an important role in a variety of physiological and psychological functions, including digestion, immune system regulation, and even mood. Further, the research shows the microbiome's intricate connection to numerous diseases, including gastrointestinal disorders, metabolic syndrome, autoimmune conditions, and even mental health issues. Consequently, modulating the microbiome has emerged as a promising therapeutic avenue for managing and preventing a wide array of health conditions. Scientists and researchers have delved into understanding the complex interplay between microbial communities and host physiology, leading to the identification of novel strategies to manipulate these communities for therapeutic purposes.

However, these traditional approaches have limitations, such as variability in individual responses, limited efficacy, and potential side effects. As a result, there has been a growing interest in developing more targeted and personalized methods for modulating the microbiome to overcome the disadvantages of the prior arts. Further, external factors such as diet, stress, and environmental exposures can have a significant impact on the composition and function of the microbiome. For example, a diet high in fiber can promote the growth of beneficial bacteria in the gut, while a diet high in sugar can promote the growth of harmful bacteria. Similarly, stress can disrupt the balance of the microbiome and lead to a range of negative health outcomes. Understanding the complex interactions between the microbiome and external factors is critical for developing effective strategies for modulating the microbiome to promote health and prevent disease. Such strategies may include dietary interventions, probiotics, prebiotics, or even fecal microbiota transplantation. Ultimately, by harnessing the power of the microbiome, we may be able to develop new treatments and therapies for a wide range of health conditions.

Accordingly, the present invention provides a method for modulating microbiome activity in an individual to treat diseases and disorders.

### SUMMARY OF THE INVENTION

Embodiments of the present invention relate to a method for modulating microbiome activity in an individual comprises administering an effective amount of a microbiome-modulating agent d for treating diseases and disorders. The microbiome-modulating agent is selected from the group consisting of antimicrobial agents, probiotic agents, prebiotic agents, or combinations thereof. The microbiome-modulating agent is administered orally, intranasally, intramuscularly, intravenously or topically or any other suitable route. Further, the microbiome-modulating agent is administered in an amount effective to modulate microbiome activity.

In accordance with an embodiment of the present invention, the amount of the microbiome-modulating agent administered depends on a type of agent and the condition being treated.

In accordance with an embodiment of the present invention, the method is used to treat a gastrointestinal disorder, a metabolic disease, an autoimmune disease, or an infectious disease.

In accordance with an embodiment of the present invention, the microbiome-modulating agent is an antimicrobial agent selected from the group consisting of antibiotics, antifungals, antivirals, antiparasitics, or combinations thereof.

In accordance with an embodiment of the present invention, the microbiome-modulating agent is a probiotic agent selected from the group consisting of bacteria, yeasts, fungi, or combinations thereof.

In accordance with an embodiment of the present invention, the microbiome-modulating agent is a prebiotic agent selected from the group consisting of dietary fibers, polyphenols, inulin, or combinations thereof.

In accordance with an embodiment of the present invention, the diseases and disorders include gastrointestinal disorders, metabolic diseases, autoimmune diseases, or infectious diseases.

In accordance with an embodiment of the present invention, the diseases and disorders include but are not limited to inflammatory bowel disease (IBD), type 2 diabetes, rheumatoid arthritis, Clostridium difficile infection, influenza, obesity and irritable bowel syndrome (IBS).

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 is a flowchart illustrating a method for modulating microbiome activity in an individual in accordance with an embodiment of the present invention.

It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

n the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practised with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The following brief definition of terms shall apply throughout the present invention:

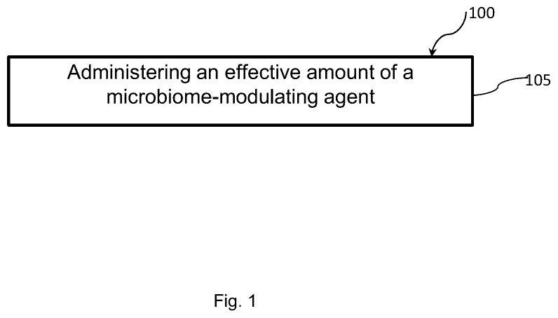

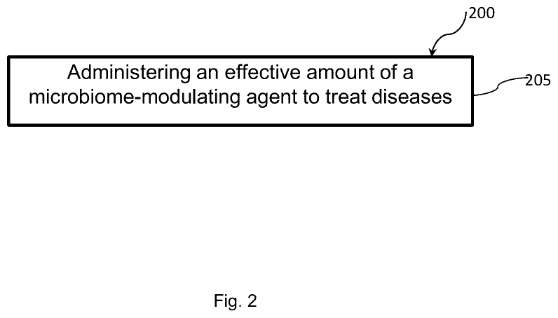

FIG. 1 and FIG. 2 are flowcharts illustrating a method for modulating microbiome activity in an individual for treating diseases and disorders in accordance with an embodiment of the present invention.

At step 105,205 an effective amount of a microbiome-modulating agent is administered into the individual in an effective amount for treating diseases and disorders. The microbiome-modulating agent is selected from the group consisting of antimicrobial agents, probiotic agents, prebiotic agents, or combinations thereof.

In particular, the administering is performed orally, intranasally, intramuscularly, intravenously or topically or any other suitable route. Further, the microbiome-modulating agent is administered in an amount effective to modulate microbiome activity.

In accordance with an embodiment of the present invention, the amount of the microbiome-modulating agent administered depends on a type of agent and the condition being treated.

In accordance with an embodiment of the present invention, the method is used to treat a gastrointestinal disorder, a metabolic disease, an autoimmune disease, or an infectious disease.

The diseases and disorders include but are not limited to inflammatory bowel disease (IBD), type 2 diabetes, rheumatoid arthritis, Clostridium difficile infection, influenza, obesity and irritable bowel syndrome (IBS) autoimmune or inflammatory disorder such as but not limited to graft-versus-host disease (GVHD), an inflammatory bowel disease (IBD), ulcerative colitis, Crohn's disease, multiple sclerosis (MS), systemic lupus erythematosus (SLE), type I diabetes, rheumatoid arthritis, Sjögren's syndrome, and Celiac disease.

In another aspect, the invention provide a method of treating or preventing a disorder associated with a distal dysbiosis in a subject in need thereof, comprising administering to the subject a probiotic composition comprising an isolated bacterial population in an amount sufficient to alter the microbiome at a site distal to the site of administration, engraftment, or colonization, such that the disorder associated with the distal dysbiosis is treated.

In one embodiment of the foregoing aspect, the disorder associated with distal dysbiosis is an autoimmune or inflammatory disease. In one embodiment of the foregoing aspect, the autoimmune or inflammatory disease is selected from the group consisting of graft-versus-host disease (GVHD), an inflammatory bowel disease (IBD), ulterative colitis, Crohn's disease, multiple sclerosis (MS), systemic lupus erythematosus (SLE), type I diabetes, rheumatoid arthritis, Sjögren's syndrome, and Celiac disease.

The formulations can be administered by any route suitable for the delivery of disclosed compositions for treating, inhibiting, or preventing a dysbiosis, or diseases and disorders associated with a dysbiosis, promoting a healthy gut flora, improving digestion, reducing inflammation, and/or promoting a healthy immune system including, but are not limited to orally, sublingually, rectally, parentally (e.g., intravenous injection (i.v.), intracranial injection (i.e.); intramuscular injection (i.m.), intraperitoneal injection (i.p.), and subcutaneous injection (s.c.) and intraosseous infusion (i.o.)), transdermally (using any standard patch), extracorporeally, inhalation, topically or the like, including topical intranasal administration or administration by inhalant. The compositions and dosage forms described herein can be administered by e.g., intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), intravesical, intrapulmonary, intraduodenal, intragastrical, intrabronchial, etc. In preferred embodiments, the pharmaceutical compositions and dosage forms described herein are administered by routes selected from oral, topical, (trans)dermal, (intra)nasal, and rectal. In certain embodiments, the (intra)nasal administration is achieved via aerosol or inhalation.

In another embodiment of the foregoing aspect, the human subject administered with a microbiome-modulating agent has dysbiosis. The dysbiosis may be a gastrointestinal dysbiosis or a distal dysbiosis.

In some embodiments of the foregoing aspect, the administration of the formulation at a first site reduces inflammation at a distal site such as blood, skin, vagina, liver, spleen, fallopian tubes, uterus, or a combination thereof in the human subject.

In one embodiment of the foregoing aspect, the disorder associated with distal dysbiosis is a transplant disorder. In one embodiment of the foregoing aspect, the transplant disorder is graft-versus-host-disease. The subject is receiving a hematopoietic stem cell transplant, a bone marrow transplant, or a solid organ transplant. In one embodiment of the foregoing aspect, the solid organ transplant is selected from the group consisting of a kidney transplant, a heart transplant, a lung transplant, a skin transplant, a liver transplant, a pancreas transplant, an intestinal transplant, an endocrine gland transplant, a bladder transplant, and a skeletal muscle transplant.

In one embodiment of the foregoing aspect, the subject has a dysbiosis at a distal site selected from the group consisting of blood, skin, vagina, liver, spleen, fallopian tubes, uterus, and combinations thereof.

In embodiments of the foregoing aspects, the microbiome-modulating agent comprises live microbial species. The live microbial species comprise live microbes, microbes that are lyophilized, freeze-dried, and/or substantially dehydrated, or the composition may comprise bacterial or fungal spores or virion.

In another embodiment, the microbiome-modulating agent comprises non-live microbial species, such as bacterial lysates or bacterial-derived products. These formulations offer stability and convenience, particularly in situations where live microbiome-modulating agent may not be feasible or practical, such as in shelf-stable supplements or processed foods.

In one embodiment, the microbiome-modulating agent comprises a single microbial species. In alternative embodiments the microbiome-modulating agent comprises a combination of microbial species. Each microbial species may be purified from a fecal material obtained from a single human subject, or from two or more human subjects.

In an embodiment of the foregoing aspects, the microbial species are selected based on their ability to treat or prevent a particular condition associated with dysbiosis of the microbiome, selected from the group consisting of obesity, diabetes, inflammatory bowel disease, and mental health disorders.

In another embodiment of the foregoing aspects, the microbial species are selected based on their ability to colonize the subject's microbiome and restore balance to the microbiome. Further, the microbial species can be selected based on their ability to produce metabolites that promote health, their ability to compete with harmful bacteria, or their ability to modulate immune function.

In yet another embodiment, the microbial species are selected based on their ability to treat or prevent a particular condition associated with dysbiosis of the microbiome. For example, the composition can be formulated to treat or prevent conditions such as obesity, diabetes, inflammatory bowel disease, and mental health disorders.

In accordance with an embodiment of the present invention, the microbiome-modulating agent is a probiotic agent selected from the group consisting of bacteria, yeasts, fungi, or combinations thereof. Probiotic agents are live microorganisms that can stimulate the growth of beneficial microorganisms in the body and/or reduce the growth of pathogenic microorganisms.

In some embodiments, the microbial species are cultured and propagated in vitro prior to administration to the subject. Moreover, the microbial species are administered to the subject in their natural state, through the consumption of fermented foods. Further, the microbial species are selected based on their ability to colonize the subject's microbiome and restore balance to the microbiome.

In accordance with an embodiment of the present invention, the microbiome-modulating agent is a slow-release probiotic formulation formulated to slowly release the one or more microbial strains over a period of time. For example, the probiotic formulation in a capsule form may be designed to release the microbial strains gradually in the gastrointestinal tract, promoting prolonged colonization and modulation of the microbiome.

Alternatively, the microbiome-modulating agent may be a fast release probiotic formulation formulated to release the one or more microbial strains over a short period of time.

In an embodiment of the foregoing invention, the probiotic formulation may be administered orally, nasally, rectally and/or vaginally.

In an exemplary example, the probiotic nasal spray formulation containing selected microbial strains, such as lactic acid bacteria and bacilli, aimed at modulating the nasal and respiratory microbiome. This formulation is designed to promote respiratory health, reduce the risk of infections, and alleviate symptoms of allergic rhinitis.

In another exemplary example, rectal suppository probiotic formulation comprising microbial strains specifically chosen for their ability to colonize the lower gastrointestinal tract effectively. This formulation is intended for individuals with gastrointestinal issues such as inflammatory bowel disease or irritable bowel syndrome, aiming to restore balance to the gut microbiome and alleviate symptoms.

For oral administration the formulation can be formulated as but not limited to a capsule, tablet, liquid, powder, or any other suitable form.

In an exemplary example, the probiotic capsule contains a single microbial species that are selected for its efficacy in treating a specific condition, such as Lactobacillus acidophilus for vaginal health or Bifidobacterium infantis for irritable bowel syndrome.

In another exemplary example, the microbiome-modulating agent formulated for oral administration comprises the microbial strains present in an amount effective to alter the microbiome of the human subject to whom the probiotic composition is orally administered. The microbiome-modulating agent may be administered to the human subject, in one or more doses, as an oral nutritional supplement.

In another exemplary example, the microbiome-modulating agent is a pharmaceutical formulation that is administered orally, nasally, rectally and/or vaginally.

In accordance with another embodiment of the present invention, the microbiome-modulating agent can be administered orally, topically, or through any other routes of administration known or to be developed in future.

In an exemplary example, a topical probiotic cream may contain microbial strains selected for their ability to promote skin health and combat conditions such as acne, eczema, or psoriasis. This formulation delivers live probiotic bacteria directly to the skin, where they can modulate the local microbiome and reduce inflammation.

In an embodiment of the foregoing aspect, the formulation can also be formulated as a food product.

In another aspect, the food product may be a medical food product or a non medical food product.

In some embodiments of the foregoing aspects, the food product is an infant formula.

In some embodiments of the foregoing aspects, the food product is a yogurt. In some embodiments of the foregoing aspects, the food product is a beverage, e.g., chilled beverage.

In some embodiments of the foregoing aspects, the composition is administered daily through the consumption of a food product comprising the pharmaceutical product.

In accordance with an embodiment of the present invention, the microbiome-modulating agent is a prebiotic agent selected from the group consisting of dietary fibers, polyphenols, inulin, or combinations thereof. Prebiotic agents are non-digestible food ingredients that stimulate the growth of beneficial microorganisms in the body.

Prebiotic is an ingredient that allows specific changes, both in the composition and/or activity in the gastrointestinal microbiota that may (or may not) confer benefits upon the host. In some embodiments, a prebiotic can be a comestible food or beverage or ingredient thereof. In some embodiments, a prebiotic may be a selectively fermented ingredient. Prebiotics may include complex carbohydrates, amino acids, peptides, minerals, or other essential nutritional components for the survival of the bacterial composition. Prebiotics include, but are not limited to, amino acids, biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carregenaan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharide.

The microbiome-modulating agent is selected from the group consisting of dietary fibers, oligosaccharides, polyphenols, lactic acid bacteria, bifidobacteria, butyrate-producing bacteria, bacteriophages, and microbial metabolites. Further, the microbiome-modulating agent is present in an effective amount to modulate the microbiome of the subject.

In accordance with an embodiment of the present invention, the microbiome-modulating agent is an antimicrobial agent selected from the group consisting of antibiotics, antifungals, antivirals, antiparasitics, or combinations thereof. Antimicrobial agents can be used to reduce the growth of pathogenic microorganisms in the body and/or to reduce inflammation.

In embodiments of the foregoing aspects, the microbiome-modulating agent comprises live microbial species. The live microbial species comprise live microbes, microbes that are lyophilized, freeze-dried, and/or substantially dehydrated, or the composition may comprise bacterial or fungal spores or virion.

In another embodiment, the microbiome-modulating agent comprises non-live microbial species, such as bacterial lysates or bacterial-derived products.

In one embodiment, the microbiome-modulating agent comprises a single microbial species. In alternative embodiments the microbiome-modulating agent comprises a combination of microbial species.

In an embodiment of the foregoing aspects, the microbial species are selected based on their ability to treat or prevent a particular condition associated with dysbiosis of the microbiome, selected from the group consisting of obesity, diabetes, inflammatory bowel disease, and mental health disorders.

In accordance with alternative embodiment of the present invention, the microbiome-modulating agent may include one or more microbiome-modulating agent selected from prebiotics, postbiotics, synbiotics, or combinations thereof, a carrier suitable for delivery of the microbiome-modulating agent to the subject.

Also provided are methods of treating or preventing a microbiome-associated disorder including but not limited to inflammatory bowel disease, irritable bowel syndrome, obesity, diabetes, autoimmune diseases, allergies, and neurological disorders in a subject by administering the formulation in an amount that alleviates symptoms associated with the microbiome-associated disorder.

Also provided are methods of treating or preventing a mammalian subject suffering from or at risk of developing a metabolic disease, and disorder or condition selected from the group consisting of diabetes, metabolic syndrome, obesity, heart disease, autoimmune disease, liver disease, and autism using the therapeutic compositions provided herein.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A method for modulating microbiome activity in an individual comprising step of:
administering an effective amount of a microbiome-modulating agent selected from the group consisting of antimicrobial agents, probiotic agents, prebiotic agents, or combinations thereof,
wherein the administering is performed orally, intranasally, intramuscularly, intravenously or topically or any other suitable route,
wherein said microbiome-modulating agent is administered in an amount effective to modulate microbiome activity.

2. The method as claimed in claim 1, wherein the amount of the microbiome-modulating agent administered depends on a type of agent and the condition being treated.

3. The method of claim 1, wherein the method is used to treat a gastrointestinal disorder, a metabolic disease, an autoimmune disease, or an infectious disease.

4. The method as claimed in claim 1, wherein the microbiome-modulating agent is an antimicrobial agent selected from the group consisting of antibiotics, antifungals, antivirals, antiparasitics, or combinations thereof.

5. The method as claimed in claim 1, wherein the microbiome-modulating agent is a probiotic agent selected from the group consisting of bacteria, yeasts, fungi, or combinations thereof.

6. The method as claimed in claim 1, wherein the microbiome-modulating agent is a prebiotic agent selected from the group consisting of dietary fibers, polyphenols, inulin, or combinations thereof.

7. The method as claimed in claim 1, wherein said diseases and disorders include gastrointestinal disorders, metabolic diseases, autoimmune diseases, or infectious diseases.

8. The method as claimed in claim 1, wherein the diseases and disorders include inflammatory bowel disease (IBD), type 2 diabetes, rheumatoid arthritis, Clostridium difficile infection, influenza, obesity and irritable bowel syndrome (IBS).

9. A method for treating diseases and disorders in an individual comprising administering an effective amount of a microbiome-modulating agent to treat said diseases and disorders;
wherein the microbiome-modulating agent is selected from the group consisting of antimicrobial agents, probiotic agents, prebiotic agents, or combinations thereof;
wherein the administering is performed orally, intranasally, intramuscularly, intravenously or topically or any other suitable route; and
wherein said microbiome-modulating agent is administered in an amount effective to modulate microbiome activity.

10. The method as claimed in claim 1, wherein said diseases and disorders include gastrointestinal disorders, metabolic diseases, autoimmune diseases, or infectious diseases.

11. The method as claimed in claim 1, wherein the diseases and disorders include inflammatory bowel disease (IBD), type 2 diabetes, rheumatoid arthritis, Clostridium difficile infection, influenza, obesity and irritable bowel syndrome (IBS).

12. The method as claimed in claim 1, wherein the amount of the microbiome-modulating agent administered depends on a type of agent and the condition being treated.

13. The method of claim 1, wherein the method is used to treat a gastrointestinal disorder, a metabolic disease, an autoimmune disease, or an infectious disease.

14. The method as claimed in claim 1, wherein the microbiome-modulating agent is an antimicrobial agent selected from the group consisting of antibiotics, antifungals, antivirals, antiparasitic, or combinations thereof.

15. The method as claimed in claim 1, wherein the microbiome-modulating agent is a probiotic agent selected from the group consisting of bacteria, yeasts, fungi, or combinations thereof.

16. The method as claimed in claim 1, wherein the microbiome-modulating agent is a prebiotic agent selected from the group consisting of dietary fibers, polyphenols, inulin, or combinations thereof.
